(19) United States
(12) Patent Application Publication
(10) Pub. No.: US 2005/0244381 A1
(43) Pub. Date: Nov. 3, 2005

(54) Adenovirus including a gene coding for a superoxide dismutase

(75) Inventors: Jacques Mallet, Paris (FR); Frederic Revah, Paris (FR); Michel Perricaudet, Ecrosnes (FR); Martine Barkats, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: AVENTIS PHARMA S.A.

(21) Appl. No.: 11/153,317

(22) Filed: Jun. 16, 2005

Related U.S. Application Data
(62) Division of application No. 08/765,026, filed on Jan. 13, 1997.

(30) Foreign Application Priority Data
Jun. 29, 1994 (FR) 94/08029

(51) Int. Cl. A61K 48/00; C12N 15/861; C12N 7/00

(52) U.S. Cl. 424/93.2; 514/44; 435/456; 435/235.1

(57) ABSTRACT

A defective recombinant adenovirus including at least one DNA sequence coding for all or an active part of a superoxide dismutase or a derivative thereof. The therapeutical use thereof and corresponding pharmaceutical compositions are also disclosed.

The present invention relates to recombinant adenoviruses which encompass a DNA sequence encoding a superoxide dismutase, and to its uses in gene therapy.

Oxygen occupies an essential position in numerous physiological or pathological processes. The reduction of molecular oxygen gives rise to the formation of highly reactive chemical species such as the superoxide radical, hydrogen peroxide and the hydroxyl radical. This latter, which is formed from superoxide and hydrogen peroxide by the Haber-Weiss reaction, is the most reactive free radical. Due to the presence of a free electron in their external layer, these radicals are highly reactive. This reactivity can be harmful to important biological molecules such as DNA, essential cellular proteins and membrane lipids. Furthermore, these free radicals can initiate chain reactions, such as lipid peroxidation, which can impair the integrity of the cells and cause their destruction.

A series of antioxidant defence mechanisms exists naturally for the purpose of regulating this production of free radicals and preventing damage to tissues and/or cells.

Thus, formation of these highly reactive entities is normally regulated or inhibited by dismutation of the superoxide ion, by means of the enzyme superoxide dismutase, to form hydrogen peroxide, with this latter then being converted into water and oxygen either by glutathione peroxidase or catalase.

Unfortunately, these regulatory mechanisms are not completely effective under certain conditions. This results in an excess of free radicals, leading to pathologies of the inflammation, emphysema, neoplasm or retinopathy type. Thus, it is nowadays recognized that these free radicals are involved in atherosclerosis, cardiovascular diseases, cirrhosis of the liver, diabetes, cataract formation, in a certain number of neurological diseases including Parkinson's disease and cerebral ischemia, in trisomy 21, and also in the ageing process. Lastly, the superoxide anion also appears to be involved in the pathogenesis of pulmonary hypertension which is induced by TNF (tumour necrosis factor).

To be more precise, the object of the present invention is to propose a means for compensating for this type of deficiency in the natural regulatory mechanisms by means of intervening, more specifically, in relation to the activity of superoxide dismutase.

As previously explained, the principal function of this enzyme, in mammals, is to destroy the superoxide radicals which are generated in various biological oxidoreduction reactions. Consequently, this enzyme is particularly important since it provides a defence against oxygen toxicities and any damage which can be caused to the cells by carcinogenic hydrocarbons.

In fact, superoxide dismutase represents a variety of different enzymes which are present in the majority of living organisms. Three forms of SOD exist, each of which has a distinctive distribution and is characterized by the nature of its metal constituent: intracellular CuZnSOD which is specific for eucaryotes, MnSOD which is dependent on manganese and is produced within the mitochondria of eucaryotes and procaryotes (Creagan R. et al. Humangenetic 20 203-209 1973) and cytosolic FeSOD, which is dependent on iron and is mainly present in procaryotes (Hendrickson D et al. Genomics 8, 736-738 1990). An extracellular copper and zinc SOD also exists.

The intracellular CuZn superoxide dismutase, termed SOD1, constitutes approximately 85 to 90% of all cellular SOD activity. It is a dimeric protein which is apparently composed of two identical subunits which are bound non-covalently to each other and each of which has a molecular weight in the order of 16,000 to 19,000 (Lieman-Burwitz J. et al.; Biochem Int. 3:107-115, 1981). The locus for human cytoplasmic 25 superoxide dismutase is present on chromosome 21. (Tan Y. H. et al. J. Exp. Med. 137: 317-330, 1973).

Normally, endogenous CuZn superoxide dismutase is present in the tissues in limited quantities and its concentration proves to be clearly inadequate when substantial quantities of superoxide anions are produced.

Furthermore, it was recently demonstrated that point mutations in the human CuZnSOD gene were associated with the development of a pathology, amyotrophic lateral sclerosis (ALS). This serious disease involves lethal degeneration of the motor neurones in the brain and the spinal cord. These mutations affect the activity of the corresponding enzyme CuZnSOD (Deng H. X. et al., Science, 261, 1047 1993).

There is, therefore, currently a requirement for an exogenous CuZnSOD which can be administered clinically in order to compensate for such deficiencies or anomalies.

Conversely, too high a concentration of SOD can, under certain conditions, be toxic to the cells which produce it. SOD is a protective enzyme which normally ensures a minimal level of superoxide radicals within the cell. In order to do this, it catalyses the interaction of free radicals so as to oxidize the one and reduce the other, that is a dismutation reaction which leads to the formation of hydrogen peroxide. In itself, the superoxide radical is not particularly toxic. The danger comes from its ability to interact with hydrogen peroxide to generate singlet oxygen and hydroxyl radicals, two forms of oxygen which are highly reactive and extremely toxic. An increased quantity of superoxide dismutase can therefore lead to an increased production of hydrogen peroxide with the previously explained consequences. This phenomenon is expressed physiologically, in particular, by an increase in lipoperoxidation accompanied by a decrease in the content of unsaturated fatty acid in the cell membranes and, as the main consequence, disruption of the membrane functions.

It would, therefore, be advantageous, in this latter case, to be able to regulate the activity of superoxide dismutase either by using an antisense sequence, for example, or dominant negative mutants.

Consequently, the clinical potential of the enzyme superoxide dismutase is considerable and it would be particularly important to be able effectively to control its activity either by stimulating it, suppressing it or compensating for it.

More specifically, the present invention relates to the development of vectors which are particularly efficacious for delivering, in vivo and in a localized manner, therapeutically active quantities of the specific gene encoding a superoxide dismutase or one of its derivatives.

The co-pending application No. PCT/EP93/02519 demonstrated that it was possible to use adenoviruses as vectors for transferring a foreign gene in vivo into the nervous system and expressing the corresponding protein.

The present invention relates, more particularly, to novel constructs which are particularly suitable and efficacious for controlling the expression of superoxide dismutase.

More specifically, it relates to a recombinant adenovirus which encompasses a DNA sequence which is suitable for controlling the expression of superoxide dismutase, to its preparation and to its use in therapeutic treatments and/or the prevention of various pathologies.

Thus, the applicant has demonstrated that it is possible to construct recombinant adenoviruses which contain a sequence encoding a superoxide dismutase, and to administer these recombinant adenoviruses in vivo, and that this administration makes it possible to achieve stable and localized expression of therapeutically active quantities of superoxide dismutase in vivo.

A first subject of the invention is therefore a defective recombinant-adenovirus which encompasses at least one DNA sequence encoding all or an active part of a superoxide dismutase or one of its derivatives.

The superoxide dismutase produced within the scope of the present invention can be a human or animal superoxide dismutase. According to one preferred embodiment of the invention, the superoxide dismutase is one of the three forms of human superoxide dismutase which were previously described, i.e. CuZnSOD (SOD₃), MnSOD (SOD₂) and extracellular SOD (SOD₃). More preferably, the DNA sequence which is integrated into the adenovirus according to the invention encodes all or an active part of human intracellular CuZn superoxide dismutase, hSOD1, or one of its derivatives.

The DNA sequence which encodes superoxide dismutase and which is employed within the scope of the present invention can be a cDNA, a genomic DNA (GDNA) or a hybrid construct consisting, for example, of a cDNA into which one or more introns are inserted. The DNA sequence can also consist of synthetic or semisynthetic sequences.

A cDNA or a gDNA is particularly is advantageously employed.

According to a preferred embodiment of the invention, the DNA sequence is a genomic DNA sequence (gDNA) which encodes a superoxide dismutase. Its use can make it possible to achieve improved expression in human cells.

Naturally, the DNA sequence can, prior to its incorporation into an adenovirus vector according to the invention, be advantageously modified, for example by site-directed mutagenesis, particularly in order to insert appropriate restriction sites. Thus, the sequences described in the prior art are not constructed for use in accordance with the invention and prior adaptations can prove to be necessary in order to obtain significant expression.

Within the meaning of the present invention, a derivative of superoxide dismutase is understood to mean any sequence which is obtained by modification and which encodes a product which retains at least one of the biological properties of superoxide dismutase. Modification is understood to mean any mutation, substitution, deletion, addition or modification of a genetic and/or chemical nature. These modifications can be effected using the techniques known to the person skilled in the art (see general molecular biological techniques below). The derivatives within the meaning of the invention can also be obtained by means of hybridization from nucleic acid libraries using the native sequence, or a fragment thereof, as a probe.

These derivatives are, in particular, molecules which have a greater affinity for their binding sites, sequences which allow improved expression in vivo, molecules which display greater resistance to proteases, and molecules which have a greater therapeutic efficacy or fewer side effects or, where appropriate, novel biological properties.

Those preferred derivatives which may more particularly be cited are natural variants, molecules in which one or more residues have been substituted, derivatives obtained by deleting regions which are not involved, or are only involved to a slight extent, in the interaction with the binding sites under consideration or which express an undesirable activity, and derivatives which include, as compared with the native sequence, additional residues such as, for example, a secretory signal and/or a junction peptide.

The scope of the present invention is also understood to cover, by means of the term derivative of superoxide dismutase, mutants which are referred to as dominant negative mutants of superoxide dismutase. More specifically, the cloned gene is in this case altered such that it encodes a mutant product which is able to inhibit the cellular activity of the wild-type superoxide dismutase. This type of derivative is particularly advantageous when, for example, attempting to suppress natural overexpression of the superoxide dismutase.

The DNA sequence which encodes all or part of the superoxide dismutase or one of its derivatives can also be an antisense sequence whose expression in the target cell makes it possible to control expression of the superoxide dismutase. Preferably, the heterologous DNA sequence includes a gene which encodes an antisense RNA which is able to control translation of the corresponding mRNA. The antisense sequence can be all or only a part of the DNA sequence which encodes the superoxide dismutase, which sequence is inserted in the opposite orientation in the vector according to the invention.

According to one particular embodiment of the invention, the DNA sequence which encodes the superoxide dismutase or one of its derivatives also includes a secretory signal which enables the synthesized superoxide dismutase to be directed into the secretory pathways of the infected cells. In this way, the synthesized superoxide dismutase is advantageously liberated into the extracellular compartments. However, the secretory signal can also be a heterologous secretory signal or even an artificial secretory signal. In the specific case of the SOD₃ form, the secretory signal can advantageously be the native SOD₃ signal.

The sequence encoding superoxide dismutase is advantageously placed under the control of signals which enable it to be expressed in the target cells. Preferably, these signals are heterologous expression signals, that is signals which are different from those which are naturally responsible for expressing the superoxide dismutase. They can, in particular, be sequences which are responsible for expressing other proteins, or else synthetic sequences. In particular, they can be promoter sequences from eucaryotic or viral genes. For example, they can be promoter sequences which are derived from the genome of the cell which it is desired to infect. Similarly, they can be promoter sequences which are derived from the genome of a virus including the adenovirus which is employed. Examples which may be cited in this respect are the promoters E1A, MLP, CMV, RSV-LTR, etc. Moreover, these expression sequences can be modified by adding activating sequences, regulatory sequences or sequences which permit tissue-specific expression. Thus, it can be particularly advantageous to employ expression signals which are specifically active, or in the main active, in the target cells such that the DNA sequence is only expressed and only produces its effect when the virus has actually infected a target cell.

In a first particular embodiment, the invention relates to a defective recombinant adenovirus which encompasses a cDNA sequence encoding human intracellular CuZn superoxide dismutase under the control of the RSV-LTR promoter.

In another particular embodiment, the invention relates to a defective recombinant adenovirus which encompasses a gDNA sequence encoding human intracellular CuZn superoxide dismutase under the control of the RSV-LTR promoter..

A particularly preferred embodiment of the present invention resides in a defective recombinant adenovirus which encompasses the ITR sequences, an encapsidation sequence, and a DNA sequence encoding human intracellular CuZn superoxide dismutase, or a derivative thereof, under the control of a promoter permitting preponderant expression in the target tissues, and in which the E1 gene and at least one of the genes E2, E4 and L1-L5 is non-functional.

The defective adenoviruses according to the invention are adenoviruses which are unable to replicate autonomously within the target cell. In general, the genome of the defective adenoviruses employed within the scope of the present invention therefore lacks at least sequences which are necessary for replication of the said virus within the infected cell. These regions can either be eliminated (in whole or in part) or rendered non-functional or replaced by other sequences and, in particular, by the DNA sequence which encodes superoxide dismutase.

Preferably, the defective virus of the invention retains its genome sequences which are required for encapsidating the viral particles. Still more preferably, as indicated above, the genome of the defective recombinant virus according to the invention encompasses the ITR sequences, an encapsidation sequence, and the non-functional E1 gene and at least one of the genes E2, E4 and L1-L5 which is/are non-functional.

Different serotypes of adenovirus exist, whose structure and properties vary somewhat. Of these serotypes, preference is given, within the scope of the present invention, to employing human type 2 or type 5 adenoviruses (Ad 2 or Ad 5) or adenoviruses of animal origin (see application FR 93 05954). Those adenoviruses of animal origin which can be employed within the scope of the present invention and which may be cited are adenoviruses of canine, bovine, murine, (example: Mav1, Beard et al., Virology 75 (1990) 81), ovine, porcine, avian and also simian (example: SAV) origin. Preferably, the adenovirus of animal origin is a canine adenovirus, more preferably a CAV2 adenovirus [Manhattan strain or A26/61 (ATCC VR-800) for example]. Preferably, use is made, within the scope of the invention, of adenoviruses of human or canine origin, or of a mixture of these viruses.

The defective recombinant adenoviruses according to the invention can be prepared by any technique known to the person skilled in the art (Levrero et al., Gene 101 (1991) 195, EP 185 573; Graham, EMBO J. 3 (1984) 2917). In particular, they can be prepared by homologous recombination between an adenovirus and a plasmid which carries, inter alia, the DNA sequence encoding superoxide dismutase. The homologous recombination takes place after cotransfection of the said adenovirus and plasmid into an appropriate cell line. The cell line which is employed should preferably (i) be transformable by the said elements, and (ii) contain the sequences which are able to complement the defective adenovirus genome part, preferably in an integrated form in order to avoid the risk of recombination. As an example of a cell line, mention may be made of the human embryonic kidney line 293 (Graham et al., J. Gen. Virol. 36 (1977) 59) which contains, in particular, integrated into its genome, the left-hand part of the genome of an Ad5 adenovirus (12%). Strategies for constructing vectors derived from adenoviruses have also been described in applications Nos. FR 93 05954 and FR 93 08596, which are incorporated into the present application by reference.

Afterwards, the adenoviruses which have replicated are recovered and purified using conventional molecular biological techniques.

The properties of the vectors of the invention which are particularly advantageous ensue, in particular, from the construct employed (defective adenovirus in which certain viral regions are deleted), from the promoter which is employed for expressing the sequence encoding superoxide dismutase (preferably a viral or tissue-specific promoter), and from the methods of administering the said vector, resulting in an expression of superoxide dismutase which is efficient and which takes place in the appropriate tissues.

The present invention also relates to any employment of an adenovirus such as described above for preparing a pharmaceutical composition which is intended for treating and/or preventing the previously cited pathologies. More particularly, it relates to any employment of these adenoviruses for preparing a pharmaceutical composition which is intended for treating and/or preventing neurodegenerative diseases such as, for example, Parkinson's disease, Alzheimer's disease, amyotrophic lateral sclerosis (ALS), and 21 trisomy. They can also be advantageously employed in the treatment of atherosclerosis, of cardiovascular diseases, of cirrhosis of the liver, of diabetes, of cataract formation, and of the ageing process.

It is, moreover, perfectly possible to envisage jointly administering an adenovirus according to the invention and at least one second adenovirus containing a gene encoding catalase (P. Amstad et al. Biochemistry 1991, 30, 9305-9313), which is another enzyme which is important in the regulation of free radical production.

The present invention also relates to a pharmaceutical composition comprising at least one or more defective recombinant adenoviruses such as previously described which is/are associated, if the need arises, with a recombinant adenovirus which contains a gene encoding catalase.

These pharmaceutical compositions can be formulated with a view to administering them by the topical, oral, parenteral, intranasal, intravenous, intramuscular, subcutaneous, intraocular, transdermal, etc. route. Preferably, the pharmaceutical compositions of the invention contain an excipient which is pharmaceutically acceptable for an injectable formulation, in particular for an injection directly into the patient. These injectable formulations can, in particular, be sterile, isotonic solutions, or dry, in particular lyophilized, compositions which, when sterilized water or physiological saline, as the case ay be, are added to them, give rise to injectable solutions.

In this respect, the invention also relates to a method for treating neurodegenerative diseases, which method comprises administering a recombinant adenovirus, such as defined above, to a patient. More specifically, the invention relates to a method for treating neurodegenerative diseases, which method comprises the stereotactic administration of a recombinant adenovirus such as defined above.

The doses of defective recombinant adenovirus which are employed for the injection can be adjusted in accordance with different parameters, in particular in accordance with the mode of administration employed, the pathology concerned, or else the duration of the sought-after treatment. In general, the recombinant adenoviruses according to the invention are formulated and administered in the form of doses of between 10⁴ and 10¹⁴ pfu/ml, preferably from 10⁶ to 10¹⁰ pfu/ml. The term pfu (plaque forming unit) corresponds to the infective power of a virus solution and is determined by infecting an appropriate cell culture and then measuring, generally after 48 hours, the number of plaques on the infected cells. The techniques for determining the pfu titre of a viral solution are well documented in the literature.

The invention also relates to any mammalian cell which is infected with one or more defective recombinant adenoviruses such as described above. More specifically, the invention relates to any population of human cells which is infected with these adenoviruses. These cells can, in particular, be fibroblasts, myoblasts, hepatocytes, keratinocytes, endothelial cells, glial cells, etc.

These cells according to the invention can be derived from primary cultures. The latter can be removed by any technique known to the person skilled in the art and then cultured under conditions which permit their proliferation. Fibroblasts, more specifically, can easily be obtained from biopsies, for example using the technique described by Ham [Methods Cell. Biol. 21a (1980) 255]. These cells can either be employed directly for infection with adenoviruses, or else preserved, for example by freezing, in order to establish autologous banks which can be used at a later date. The cells according to the invention can also be secondary cultures which are obtained, for example, from previously established banks.

The cells in culture are then infected with recombinant adenoviruses in order to confer on them the capacity to produce superoxide dismutase. The infection is carried out in vitro using techniques known to the person skilled in the art. In particular, the person skilled in the art can adjust the multiplicity of infection in accordance with the type of cells employed and the number of copies of the virus which are required per cell. It is, of course, understood that these steps have to be carried out under appropriate conditions of sterility when the cells are destined for in vivo administration. The doses of recombinant adenovirus which are used for infecting the cells can be adjusted by the person skilled in the art in accordance with the sought-after aim. The conditions described above for in vivo administration can be applied to in vitro infection.

The invention also relates to an implant which comprises mammalian cells, which are infected with one or more defective recombinant adenoviruses such as described above, and an extracellular matrix. Preferably, the implants according to the invention comprise from 10⁵ to 10¹⁰ cells. More preferably, they comprise from 10⁶ to 10⁸ cells. 20 More specifically, the extracellular matrix in the implants of the invention comprises a gelling compound and, where appropriate, a support for anchoring the cells.

Different types of gelling agent can be employed for preparing the implants according to the invention. The gelling agents are used in order to enclose the cells in a matrix having the constitution of a gel and, if need be, to promote anchorage of the cells to the support. Different cell adhesion agents an, therefore, be used as gelling agents, such as, in particular, collagen, gelatin, glycosaminoglycans, fibronectin, lectins, agarose, etc.

As indicated above, the compositions according to the invention advantageously include a support for anchoring the cells. The term anchoring designates any form of biological and/or chemical and/or physical interaction resulting in adhesion and/or fixation of the cells to the support. Furthermore, the cells can either cover the support which is used, or penetrate into the interior of this support, or do both. Within the scope of the invention, preference is given to using a solid, non-toxic and/or biocompatible support. In particular, it is possible to use polytetrafluoroethylene (PTFE) fibres or a support of biological origin.

The implants according to the invention can be implanted at different sites in the organism. In particular, the implantation can be carried out within the peritoneal cavity, in the subcutaneous tissue (sub-pubic region, iliac or inguinal fossae, etc.), in an organ, a muscle, a tumour, the central nervous system, or else under a mucous membrane. The implants according to the invention are particularly advantageous in that they render it possible to control the liberation of the therapeutic product within the organism: this liberation is firstly determined by the multiplicity of infection and by the number of implanted cells. subsequently, liberation can be controlled either by shrinkage of the implant, which definitively arrests the treatment, or by using expression systems which can be regulated and which make it possible to induce or suppress expression of the therapeutic genes.

The present invention thus supplies viral vectors which can be used directly in gene therapy and which are particularly suitable and efficacious for directing the expression of superoxide dismutase in vivo. The present invention thus offers a novel approach which is particularly advantageous for treating and/or preventing numerous pathologies such as those mentioned above.

Furthermore, the adenoviral vectors according to the invention exhibit substantial advantages which are associated, in particular, with their very high degree of efficacy in infecting the target cells, thereby making it possible to achieve infections using low volumes of viral suspension. Furthermore, infection with the adenoviruses of the invention is highly localized to the site of injection, thereby avoiding the risk of diffusion to adjacent cerebral structures.

This treatment can relate both to man and to any animal such as sheep, cattle, rodents, domestic animals (dogs, cats, etc.), horses, fish, etc.

The examples and the figure are presented below by way of illustrating, and not limiting, the sphere of the invention.

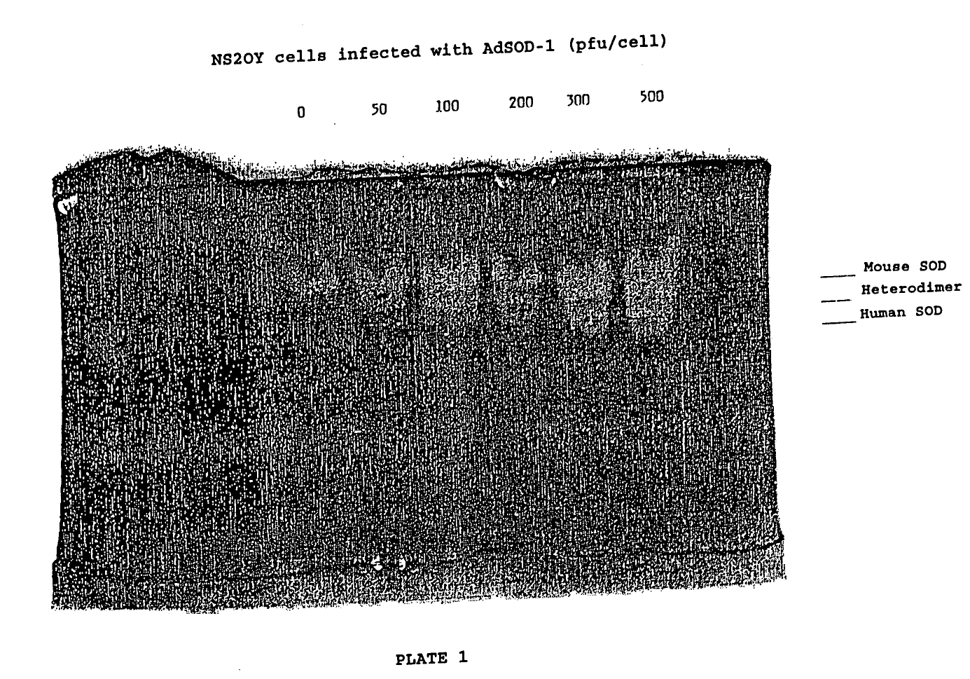

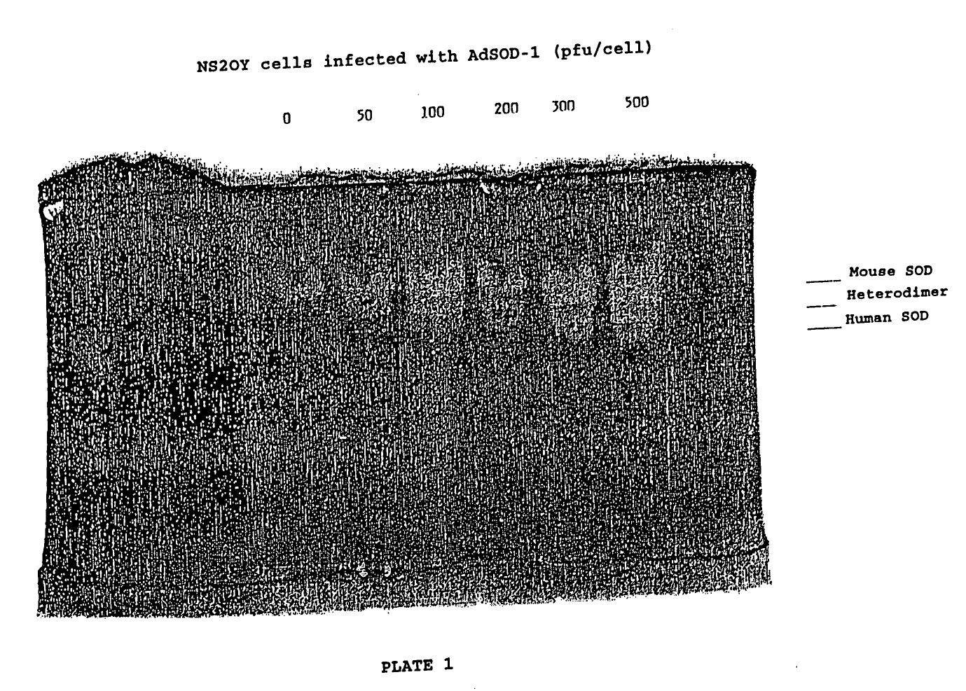

FIG. 1 : Enzymic activity of human CuZnSOD (hSOD-1) in NS2OY cells which are infected with a recombinant adenovirus encoding hSOD-1 (from 0 to 500 pfu/cell).

### GENERAL MOLECULAR BIOLOGICAL TECHNIQUES

The standard molecular biological methods employed, such as preparative extractions of plasmid DNA, centrifugation of plasmid DNA in a caesium chloride gradient, electrbphoresis on agarose or 10 acrylamide gels, purification of DNA fragments by electroelution, extraction of proteins with phenol or with phenol/chloroform, precipitation of DNA in a saline medium using-ethanol or isopropanol, transformation into Escherichia coli, etc. are well known to persons skilled in the art and are amply described in the literature [Maniatis T. et al., “Molecular Cloning, a Laboratory Manual”, Cold Spring Harbor Laboratory, Cold Spring Harbor, N.Y., 1982; Ausubel F. M. et al. (eds)i “Current Protocols in Molecular Biology”, John Wiley & Sons, New York, 1987].

The plasmids of the pBR322 and pUC type, and the phages of the M13 series are obtained commercially (Bethesda Research Laboratories).

For ligations, the DNA fragments can be separated according to their size by electrophoresis in agarose or acrylamide gels, extracted with phenol or with a phenol/chloroform mixture, precipitated using ethanol and then incubated in the presence of T4 phage DNA ligase (Biolabs) in accordance with the supplier's instructions.

Protruding 5′ ends can be filled in using the Klenow fragment of E. coli DNA polymerase I (Biolabs) in accordance with the supplier's specifications. Protruding 3′ ends are destroyed in the presence of T4 phage DNA polymerase (Biolabs), which is used in accordance with the manufacturer's instructions. Protruding 5′ ends are destroyed by careful treatment with S1 nuclease.

In vitro site-directed mutagenesis using synthetic oligodeoxynucleotides can be carried out in accordance with the method developed by Taylor et al. [Nucleic Acids Res. 13 (1985) 8749-8764] using the kit distributed by Amersham.

Enzymic amplification of DNA fragments by means of the technique termed PCR [polymerase-catalyzed chain reaction, Saiki R. K. et al., Science 230 (1985) 1350-1354; Mullis K. B. and Faloona F. A., Meth. Enzym. 155 (1987) 335-350] can be carried out using a DNA thermal cycler (Perkin Elmer Cetus) in accordance with the manufacturer's specifications.

Nucleotide sequences can be verified by means of the method developed by Sanger et al.. [Proc. Natl. Acad. Sci. USA, 74 (1977) 5463-5467] using the kit distributed by Amersham.

### EXAMPLES

### Example 1

### Protocol for Constructing the Vectors LTRIX-hSOD1, pLTRIX-hSOD1 Gly37 and pLTRIX-hSOD1 sn139

These vectors contain the sequences which encode wild-type or mutated human SOD1 under the control of the LTR of the RSV virus as well as adenovirus sequences which permit in vivo recombination.

The cDNAs which encode the different types of SOD employed are described in Rosen et al., Nature, vol. 362, 52-62, and Deng et al., Science, vol. 261, 1047-1051.

Each cDNA is-inserted into a Bluescript plasmid (Stratagene) between the PstI and HindIII sites. A polyadenylation sequence derived from SV40 was previously introduced into the XhoI site of the same plasmid. These plasmids'are named SK-hSOD-PolyA, SK-hSODgly-PolyA and SK-hSODasn-PolyA.

The vectors pLTRIX-hSOD1, pLTRIX-hSOD1gly and pLTRIX-hSOD1 are obtained by introducing an insert, obtained by cutting SK-hSOD-PolyA, SK-hSODgly-PolyA and SK-hSODasn-PolyA with KpnI and SacI (KpnI and SacI ends rendered blunt), into the EcoRV site of the plasmid pLTRIX.

### Example 2

### Construction of Recombinant Adenoviruses which Contain a Sequence Encoding Human Intracellular CuZn Superoxide Dismutase

Vector pLTRIX-hSOD1 is linearized and cotransfected together with a deficient adenoviral vector into helper cells (line 293) which supply in trans with functions encoded by-the E1 (E1A and E1B) adenovirus regions.

More precisely, the adenovirus Ad-hSOD1 was obtained by homologous recombination in vivo between the mutant adenovirus Ad-d11324 (Thi-mappaya et al., Cell 31 (1982) 543) and vector pLTRIX-hSOD1 in accordance with the following protocol: plasmid pLTRIX-hSOD1 and adenovirus Ad-d11324, linearized with the enzyme ClaI, were cotransfected into line 293 in the presence of calcium phosphate in order to allow homologous recombination to take place. The recombinant adenoviruses which were generated in this way were selected by plaque purification. Following isolation, the DNA of the recombinant adenovirus was amplified in cell line 293, resulting in a culture supernatant containing unpurified recombinant defective adenovirus at a titre of approximately 10¹⁰ pfu/ml.

The viral particles are then purified by gradient centrifugation.

### Example 3

### Monitoring the In Vitro Expression of hSOD-1

In order to do this, use is made of the protocol described by Beauchamp and Fridovitch, 1971, Ann-Biochem. Vol. 44, pp. 276-278.

In each case, an NP-40 extract is prepared from 500,000 NS20Y cells (mouse neuroblastomas) and this extract is loaded onto a non-denaturing acrylamide gel, and electrophoresis is carried out at 100 V for 3 hours.

The superoxide dismutase is located by soaking the gel in a solution of nitroblue tetrazolium (NBT) and riboflavin, and then in a solution of tetramethylethylenediamine (TEMFED). The gel is then illuminated and, under the circumstances, becomes uniformly blue except in those positions which contain superoxide dismutase (the reduced riboflavin, in the presence of TEMED, generates superoxide radicals following reoxidation in air. The superoxide radicals which are produced reduce the colourless NBT to form a blue compound (formazan). By neutralizing the superoxide radicals which are produced, the SOD will inhibit the coloured reaction and will appear as a colourless spot).

1-28. (canceled)

29. A method for treating Parkinson's Disease, comprising stereotaxically administering into the central nervous system of a Parkinson's disease patient an implant, wherein the implant comprises an extracellular matrix and human cells infected by a replication defective, recombinant adenovirus comprising a DNA sequence which encodes an intracellular, human CuZn superoxide dismutase-1 (SOD-1), wherein the DNA sequence is under the control of a signal enabling expression in a target cell and wherein said expression of said superoxide dismutase results in a treatment of Parkinson's disease.

30. The method according to claim 29, wherein the DNA sequence is a cDNA sequence.

31. The method according to claim 29, wherein the signal enabling expression in a target cell is a viral promoter.

32. The method according to claim 31, wherein the promoter is selected from the group consisting of the E1A, MLP, CMV and RSV-LTR promoters.

33. The method according to claim 29, wherein the adenovirus lacks regions of its genome which are necessary for replication in a target cell.

34. The method according to claim 29, wherein the adenovirus comprises ITR sequences and an encapsidation sequence, and wherein the E1 gene and at least one of the E2, E4 or L1-L5 genes are non-functional.

35. The method according to claim 29, wherein the adenovirus is of a type selected from the group consisting of human Ad 2, human Ad 5, and canine CAV-2.

36. The method according to claim 30, wherein the cDNA sequence encodes human intracellular CuZn superoxide dismutase-1 (hSOD1) under the control of an RSV-LTR promoter.

37. The method according to claim 29, wherein the signal enabling expression in a target cell is a promoter permitting preponderant expression in the target cell.

38. The method according to claim 34, wherein the adenovirus comprises ITR sequences and an encapsidation sequence, and wherein the E1 gene and at least one of the E4 or L1-L5 genes are non-functional.

39. The method according to claim 29, wherein the extracellular matrix comprises a gelling compound selected from the group consisting of collagen, gelatin, glucosaminoglycans, fibronectins, and lectins.

40. The method according to claim 29, wherein said extracellular matrix comprises a support for anchoring the infected cells.

41. The method according to claim 40, wherein the support comprises polytetrafluoroethylene fibers.
